(19) United States
(12) Patent Application Publication
(10) Pub. No.: US 2007/0054951 A1
(43) Pub. Date: Mar. 8, 2007

(54) Fluoro-methanesulfonyl-substituted cycloalkanoindoles and their use as prostaglandin D2 antagonists

(76) Inventors: Lianhai Li, Montreal (CA); Christian Bcaulicu, Laval (CA); Daniel Guay, Notre Dame de I'lle (CA); Claudio Sturino, L'lle Bizard (CA); Chaoyin Wang, Kirkland (CA)

Correspondence Address: MERCK AND CO., INC, P O BOX 2000, RAHWAY, NJ, 07065-0907, US

(21) Appl. No.: 10/555,223

(22) PCT Filed: May 18, 2004
(86) PCT No.: PCT/CA04/00752

§ 371 Date: Nov. 1, 2005

Related U.S. Application Data
(60) Provisional application No. 60/471,957, filed on May 20, 2003.

(51) Int. Cl. A61K 31/403; C07D 209/94

(52) U.S. Cl. 514/411; 548/444

(57) ABSTRACT

Novel cycloalkanoindole derivatives of formula (I) are antagonists of prostaglandins, and as such are useful for the treatment of prostaglandin mediated diseases.

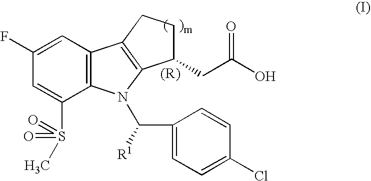

### BACKGROUND OF THE INVENTION

The present invention relates to compounds and methods for treating prostaglandin mediated diseases, and certain pharmaceutical compositions thereof. More particularly, the compounds of the invention are structurally different from steroids, antihistamines or adrenergic agonists, and are antagonists of the nasal and pulmonary congestion effects of D-type prostaglandins.

Two review articles describe the characterization and therapeutic relevance of the prostanoid receptors as well as the most commonly used selective agonists and antagonists: Eicosanoids: From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87. An article from T. Tsuri et al. published in 1997 in Journal of Medicinal Chemistry, vol 40, pp.3504-3507 states that “PGD2 is considered to be an important mediator in various allergic diseases such as allergic rhinitis, atopic asthma, allergic conjunctivitis and atopic dermatitis.” More recently, an article by Matsuoka et al. in Science (2000), 287:2013-7, describes PGD2 as being a key mediator in allergic asthma. In addition, patents such as U.S. Pat. No. 4,808,608 refer to prostaglandin antagonists as useful in the treatment of allergic diseases, and explicitly allergic asthma. PGD2 antagonists are described in, for example, European Patent Application 837,052 and PCT Application WO98/25919, as well as WO99/62555.

U.S. Pat. No. 4,808,608 discloses tetrahydrocarbazole-1-alkanoic acid derivatives as prostaglandin antagonists.

PCT Application WO0179169 discloses PGD2 antagonists having the formula:

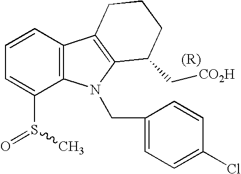

European Patent Application 468,785 discloses the compound 4-[(4-chlorophenyl)-methyl]-1,2,3,4-tetrahydro-7-(2-quinolinylmethoxy)-cyclopent[b]indole-3-acetic acid, which is a species of a genus said to be leukotriene biosynthesis inhibitors.

U.S. Pat. No. 3,535,326 discloses antiphlogistic compounds of the formula:

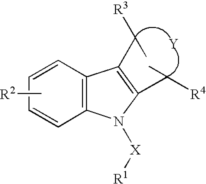

U.S. Pat. No. 6,410,583 discloses compounds of the formula:

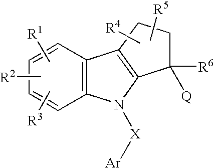

PCT Published Application WO2003062200 discloses compounds of the formula:

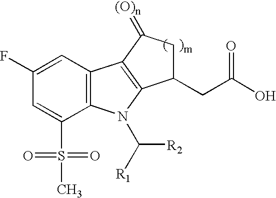

### SUMMARY OF THE INVENTION

The present invention provides novel compounds which are prostaglandin receptor antagonists; more particularly, they are prostaglandin D2 receptor (DP receptor) antagonists. Compounds of the present invention are useful for the treatment of various prostaglandin-mediated diseases and disorders; accordingly the present invention provides a method for the treatment of prostaglandin-mediated diseases using the novel compounds described herein, as well as pharmaceutical compositions containing them.

### DETAILED DESCRIPTION OF THE INVENTION

The present invention relates to compounds of formula I: 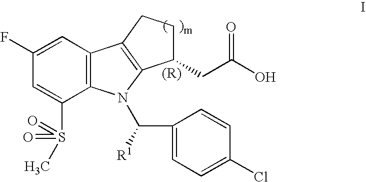
and pharmaceutically acceptable salts thereof, wherein m is 1 or 2, and R¹ is C₁₋₃alkyl optionally substituted with 1 to 5 halogen atoms.

One embodiment of formula I is the compound [(3R)-4-[(1S)-1-(4-chlorophenyl)ethyl]-7-fluoro-5-(methylsulfonyl)-1,2,3,4-tetrahydrocyclopenta[b]indol-3-yl]acetic acid and pharmaceutically acceptable salts thereof.

Another embodiment of formula I is the compound [(1R)-9-[(1S)-1-(4-chlorophenyl)ethyl]-6-fluoro-8-(methylsulfonyl)-2,3,4,9-tetrahydro-1H-carbazol-1-yl]acetic acid and pharmaceutically acceptable salts thereof.

A third embodiment of formula I is the compound [(1R)-9-[(1R)-1-(4-chlorophenyl)-2-fluoroethyl]-6-fluoro-8-(methylsulfonyl)-2,3,4,9-tetrahydro-1H-carbazol-1-yl]acetic acid and pharmaceutically acceptable salts thereof.

A fourth embodiment of formula I is the compound [(1R)-9-[(1R)-1-(4-chlorophenyl)-2,2-difluoroethyl]-6-fluoro-8-(methylsulfonyl)-2,3,4,9-tetrahydro-1H-arbazol-1-yl]acetic acid and pharmaceutically acceptable salts thereof.

Compounds of formula I are selective antagonists of the DP receptor with 10 fold or greater affinity for the DP versus other prostanoid receptor (TP, EP1, EP2, EP3, EP4, FP, IP) and the PGD2 receptor CRTH2 (also known as DP2).

In another aspect of the present invention there is provided pharmaceutical compositions comprising a compound of formula I, and a pharmaceutically acceptable carrier.

In one embodiment, the pharmaceutical compositions further comprises a second active ingredient selected from an antihistamine, a leukotriene antagonist, leukotriene biosynthesis inhibitor, prostaglandin receptor antagonists or biosynthesis inhibitors, corticosteroids, cytokine modulators, anti-IgE, anti-cholinergics or NSAIDS. In a further embodiment, the second active ingredient is selected from an antihistamine and a leukotriene antagonist. In another further embodiment, the second active ingredient is selected from montelukast, pranlukast and zafirlukast. In another further embodiment, the second active ingredient is selected from loratadine, desloratadine, fexofenadine, cetirizine, ebastine and levocetirizine.

In another aspect of the present invention there is provided a method for the treatment or prevention of prostaglandin D2 mediated diseases which comprises administering to a patient in need of treatment a therapeutically effective amount of a compound of formula I.

In one embodiment of the invention is a method of treating or preventing a prostaglandin D2 mediated disease comprising administering to a mammalian patient in need of such treatment a compound of formula I in an amount which is effective for treating or preventing a prostaglandin D2 mediated disease, wherein the prostaglandin mediated disease is nasal congestion, rhinitis including seasonal allergic rhinitis and perennial allergic rhinitis, and asthma including allergic asthma.

In another embodiment of the present invention is a method for the treatment of nasal congestion in a patient in need of such treatment which comprises administering to said patient a therapeutically effective amount of a compound of formula I.

In yet another embodiment of the present invention is a method for the treatment of asthma, including allergic asthma, in a patient in need of such treatment which comprises administering to said patient a therapeutically effective amount of a compound of formula I.

In yet another embodiment of the present invention is a method for the treatment of allergic rhinitis (seasonal and perennial) in a patient in need of such treatment which comprises administering to said patient a therapeutically effective amount of a compound of formula I.

Salts

The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

It will be understood that, unless otherwise specified, references to the compound of formula I are meant to also include the pharmaceutically acceptable salts.

Utilities

Compounds of formula I are antagonists of prostaglandin D2. The ability of compounds of formula I to interact with the DP receptor makes them useful for preventing or reversing undesirable symptoms caused by prostaglandins in a mammalian, especially human subject. The present compounds are selective for the DP receptor over the TP receptor. The antagonism of the actions of prostaglandin D2 indicates that the compounds and pharmaceutical compositions thereof are useful to treat, prevent, or ameliorate in mammals and especially in humans: respiratory conditions, allergic conditions, pain, inflammatory conditions, mucus secretion disorders, bone disorders, sleep disorders, fertility disorders, blood coagulation disorders, trouble of the vision as well as immune and autoimmune diseases. In addition, such a compound may inhibit cellular neoplastic transformations and metastic tumor growth and hence can be used in the treatment of cancer. Compounds of formula I may also be of use in the treatment and/or prevention prostaglandin D2 mediated proliferation disorders such as may occur in diabetic retinopathy and tumor angiogenesis. Compounds of formula I may also inhibit prostanoid-induced smooth muscle contraction by antagonizing contractile prostanoids or mimicking relaxing prostanoids and hence may be use in the treatment of dysmenorrhea, premature labor and eosinophil related disorders.

Accordingly, another aspect of the invention provides a method of treating or preventing a prostaglandin D2 mediated disease comprising administering to a mammalian patient in need of such treatment a compound of formula I in an amount which is effective for treating or preventing said prostaglandin D2 mediated disease. Prostaglandin D2 mediated diseases include, but are not limited to, allergic rhinitis, nasal congestion, rhinorrhea, perennial rhinitis, nasal inflammation, allergic conjunctivitis, asthma including allergic asthma, chronic obstructive pulmonary diseases and other forms of lung inflammation; pulmonary hypotension; sleep disorders and sleep-wake cycle disorders; prostanoid-induced smooth muscle contraction associated with dysmenorrhea and premature labor; eosinophil related disorders; thrombosis; glaucoma and vision disorders; occlusive vascular diseases, such as for example atherosclerosis; congestive heart failure; diseases or conditions requiring a treatment of anti-coagulation such as post-injury or post surgery treatment; rheumatoid arthritis and other inflammatory diseases; gangrene; Raynaud's disease; mucus secretion disorders including cytoprotection; pain and migraine; diseases requiring control of bone formation and resorption such as for example osteoporosis; shock; thermal regulation including fever, rejection in organ transplant and by-pass surgery, and immune disorders or conditions in which immunoregulation is desirable. More particularly the disease to be treated is one mediated by prostaglandin D2 such as nasal congestion, allergic rhinitis, pulmonary congestion, and asthma including allergic asthma.

Dose Ranges

The magnitude of prophylactic or therapeutic dose of a compound of formula I will, of course, vary with the nature and the severity of the condition to be treated and with the particular compound of formula I and its route of administration. It will also vary according to a variety of factors including the age, weight, general health, sex, diet, time of administration, rate of excretion, drug combination and response of the individual patient. In general, the daily dose from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for the oral administration of humans may contain from 0.05 mg to 5 g of active agent compounded with an appropriate and convenient amount of carrier material which may vary from about 5 to about 99.95 percent of the total composition. Dosage unit forms will generally contain between from about 0.1 mg to about 0.4 g of an active ingredient, typically 0.5 mg, 1 mg, 2 mg, 5 mg, 10 mg, 25 mg, 50 mg, 100 mg, 200 mg, or 400 mg.

Pharmaceutical Compositions

Another aspect of the present invention provides pharmaceutical compositions comprising a compound of formula I with a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

For the treatment of any of the prostanoid mediated diseases compounds of formula I may be administered orally, by inhalation spray, topically, parenterally or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffm, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing the compound of formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

Combinations with Other Drugs

For the treatment and prevention of prostaglandin mediated diseases, compound of formula I may be co-administered with other therapeutic agents. Thus in another aspect the present invention provides pharmaceutical compositions for treating prostaglandin D2 mediated diseases comprising a therapeutically effective amount of a compound of formula I and one or more other therapeutic agents. Suitable therapeutic agents for combination therapy with a compound of formula I include: (1) a prostaglandin receptor antagonist; (2) a corticosteroid such as triamcinolone acetonide; (3) a β-agonist such as salmeterol, formoterol, terbutaline, metaproterenol, albuterol and the like; (4) a leukotriene modifier, such as a leukotriene antagonist or a lipooxygenase inhibitor such as montelukast, zafirlukast, pranlukast, or zileuton; (5) an antihistamine (histamine H1 antagonist) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine, pyrilamine, astermizole, norastemizole, terfenadine, loratadine, cetirizine, levocetirizine, fexofenadine, desloratadine, and the like; (6) a decongestant including phenylephrine, phenylpropanolamine, pseudophedrine, oxymetazoline, ephinephrine, naphazoline, xylo-metazoline, propylhexedrine, or levo-desoxyephedrine; (7) an antiitussive including codeine, hydrocodone, caramiphen, carbetapentane, or dextramethorphan; (8) another prostaglandin ligand including prostaglandin F agonist such as latanoprost; misoprostol, enprostil, rioprostil, ornoprostol or rosaprostol; (9) a diuretic; (10) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (11) cyclooxygenase-2 (COX-2) inhibitors such as celecoxib and rofecoxib, etoricoxib and valdecoxib; (12) inhibitors of phosphodiesterase type IV (PDE-IV) e.g. Ariflo, roflumilast; (13) antagonists of the chemokine receptors, especially CCR-1, CCR-2, and CCR-3; (14) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (15) anti-diabetic agents such as insulin, sulfonylureas, biguanides (metformin), α-glucosidase inhibitors (acarbose) and glitazones (troglitazone, pioglitazone, englitazone, rosiglitazone and the like); (16) preparations of interferon beta (interferon beta-1a, interferon beta-1b); (17) anticholinergic agents such as muscarinic antagonists (ipratropium bromide and tiotropium bromide), as well as selective muscarinic M3 antagonists; (18) steroids such as beclomethasone, methylprednisolone, betamethasone, prednisone, dexamethasone, and hydrocortisone; (19) triptans commonly used for the treatment of migraine such as sumitriptan and rizatriptan; (20) alendronate and other treatments for osteoporosis; (21) other compounds such as 5-aminosalicylic acid and prodrugs thereof, antimetabolites such as azathioprine and 6-mercaptopurine, cytotoxic cancer chemotherapeutic agents, bradykinin (BK2 or BK1) antagonists, TP receptor antagonists such as seratrodast, neurokinin antagonists (NK1/NK2), VLA4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206.

In addition, the invention encompasses a method of treating prostaglandin D₂ mediated diseases comprising: administering to a patient in need of such treatment a therapeutically effective amount of the compound of formula I, co-administered with one or more of such ingredients as listed immediately above. The amounts of active ingredients may be those commonly used for each active ingredient when it is, administered alone, or in some instances the combination of active ingredients may result in lower dosage for one or more of the active ingredients.

The following abbreviations are used herein: AcOH=acetic acid; DCHA=dicyclohexylamine; DMAc=dimethylacetamide; DMF=dimethylformamide; DMSO=dimethyl sulfoxide; Et=ethyl; EtOAc=ethyl acetate; iPr=isopropyl; iPrOH=isopropyl alcohol; Me=methyl; MTBE-methyl t-butyl ether, rt=room temperature; THF=tetrahydrofuran; TMS=trimethylsilyl.

Compounds of Formula I of the present invention can be prepared according to the synthetic routes outlined in Schemes 1 to 6 and by following the methods described herein. Intermediate compounds of Formula VIII may be prepared by the method presented in Scheme 1 from 4-fluorophenyl hydrazine II or 2-bromo-4-fluorophenyl hydrazine XI. Reaction of II with an appropriate cycloalkanone III (where R is ester group such as an alkyl group) under Fisher Indole or similar conditions gives IV. Bromination of IV (where m=1) may be accomplished with bromine or a brominating agent such as pyridium tribromide, under basic condition in a polar solvent, for example, by carrying out the reaction in pyridine or in a solvent such as dichloromethane in the presence of pyridine followed by the mono reduction of a dibromo intermediate under acid and reducing metal conditions to generate the corresponding bromoindole VIII (where m=1). The bromoindole VIII may also be obtained from hydrazine XI by reaction with III under Fisher Indole or similar conditions.

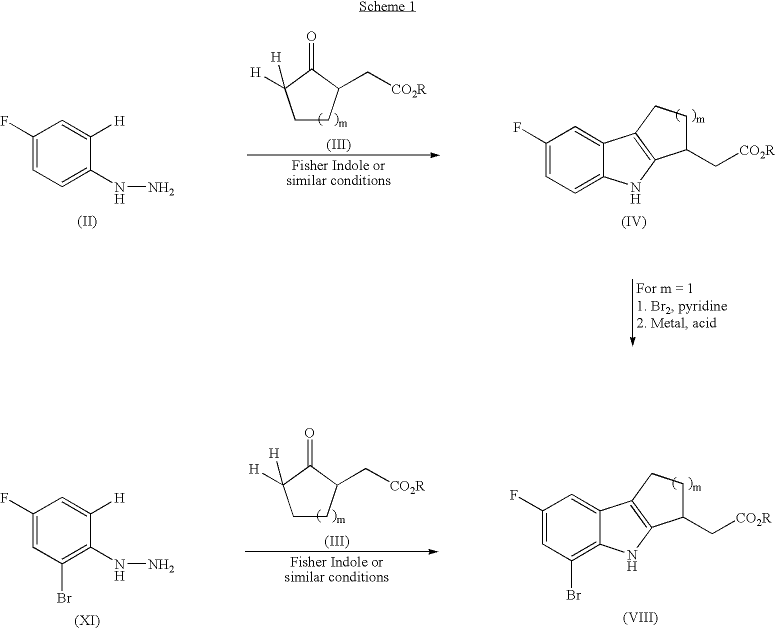

Compounds of Formula IV may alternatively be prepared by the method presented in Scheme 2 from an appropriately substituted aniline V. Condensation of V with an appropriate cycloalkanone III followed by the cyclization under Heck or similar metal catalysis conditions leads to indole IV.

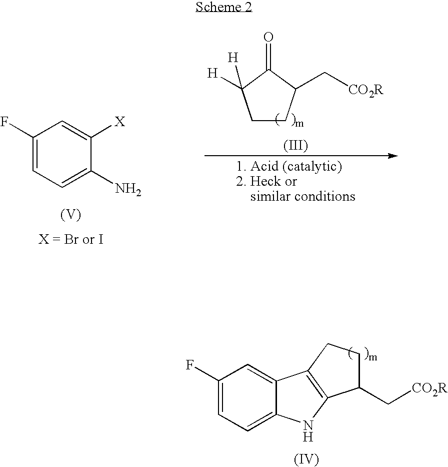

Compounds of Formula III may be prepared by the method presented in Scheme 3 from an appropriately substituted silyl enol ether VI or an appropriately substituted enamine VII. Addition of an appropriate electrophile such as Y—CH₂CO₂R (wherein Y represents a halogen or a leaving group) in the presence of a base such as an alkyl lithium or a Lewis acid such as silver trifluoroacetate with the silyl enol ether VI gives the cycloalkanone III. The compound of formula III may alternatively be prepared from the addition of Y—CH₂CO₂R on an appropriately substituted enamine VII under Stork Enamine or similar conditions.

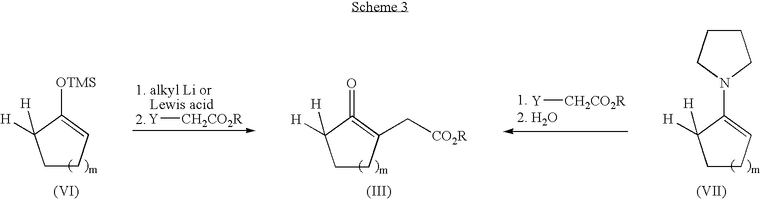

Compounds of Formula I wherein R¹ is methyl may be prepared by the method presented in Scheme 4 from bromoindole VIII. Reaction of VIII with (1R)-1-(4-chlorophenyl)ethanol under Mitsunobu conditions, i.e. in the presence of triphenylphosphine and di-t-butyl azodicarboxylate, gives N-alkylated indole IX. Coupling of IX with a methanesulfinate such as sodium methanesulfinate in the presence of Cu(I) salts leads to compounds of formula I, following ester hydrolysis. The bromoindole acid (IX, R═H) may alternatively first react with a suitable metallation agent, such as n-BuLi, followed by trapping with an electrophile such as methyl disulfide to give the corresponding methyl sulfide, which upon oxidation with, for example, hydrogen peroxide/sodium tungstate provides compound I. The steps of alkylation of the bromoindole VIII followed by sulfonylation may also be reversed; thus sulfonylation of the bromoindole VIII provides the compound X, which is alkylated using similar conditions as described before or by using Mitsunobu reaction conditions to provide compound of formula I following ester hydrolysis.

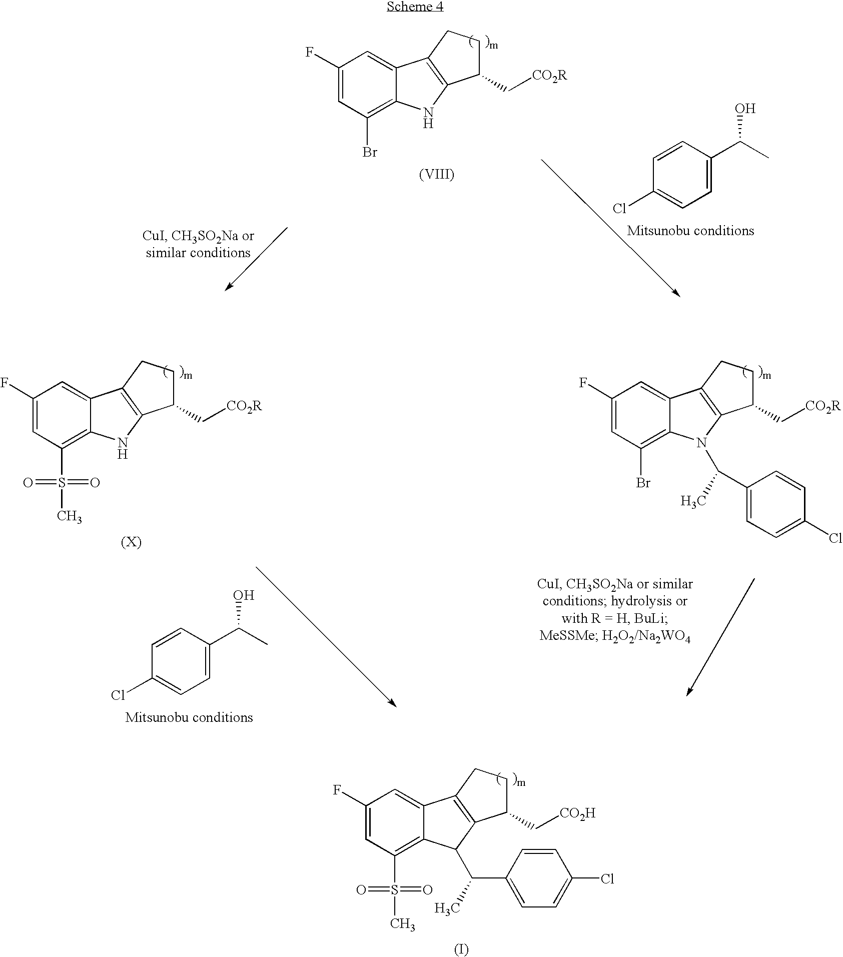

While the use of specific stereoisomer is depicted in Scheme 4, it is understood that the reactions may be carried out with racemic mixtures; resolution may be effected with any of the intermediate compound IV, VIII or X to provide the desired enantiomer. Resolution may be carried out by conventional means, for example by the use of an optically active base as a resolving agent, or by chiral separation techniques such as separation by HPLC using a chiral column. Alternatively, enzymatic resolution may be used to separate the enantiomers. For example, racemic mixture of compound (IV) where R is ethyl and m is 1, when treated with Pseudomonas fluorescens lipase is hydrolyzed to the corresponding (S)-acid, and the desired (R)-ester may then be separated and used in the preparation of the final compound. Racemic VIII may be sulfonylated as depicted in Scheme 4, and the resulting racemic X may be resolved.

In Scheme 5, racemic Xa (where m is 2 and R is ethyl) may be alkylated with the chiral reagent to provide a diastereomeric mixture of ester compound XI, which upon selective hydrolysis, provides the acid compound I with the desired stereochemistry.

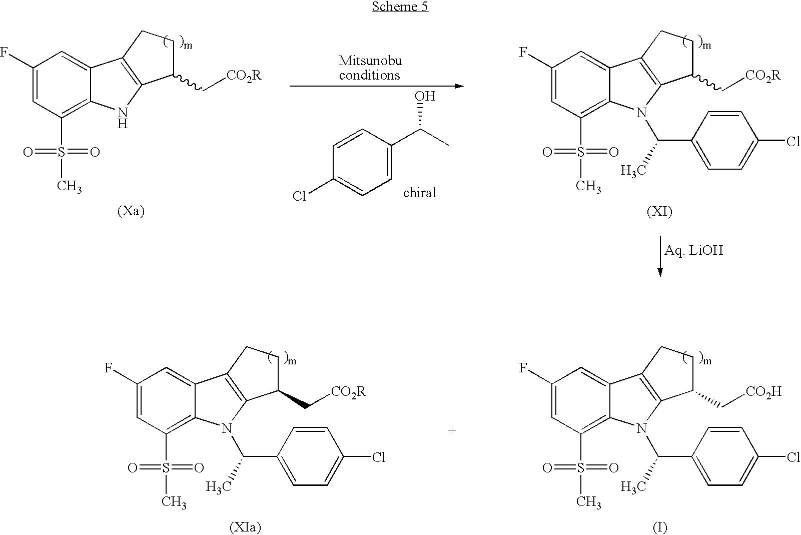

Compounds of formula I wherein R¹ is fluorinated methyl may be prepared as shown in Scheme 6. The indole X is reacted with an appropriately mono-protected diol (e.g., P=t-butyldimethylsilyl) under Mitsunobu conditions to provide the N-alkylated indole alcohol XII, following deprotection (e.g. TBAF). The indole alcohol XII is reacted with mesyl chloride followed by triethylamine trihydrofluoride to give the corresponding monofluoro compound Ia after ester hydrolysis. Oxidation of indole alcohol XII using, for example, Dess-Martin periodinane gives the corresponding aldehyde, which upon treatment with DAST (diethylaminsulfur trifluoride) provides the corresponding difluoro compound Ib after ester hydrolysis. Oxidation of XII using, for example, Dess-Martin or Swern protocol followed by aqueous hypochlorite treatment affords the corresponding carboxylic acid which may be treated with cyanuric acid or a 2-fluoropyridinium reagent or thionyl chloride and KHF₂ to give the acyl fluoride XIII. Compound of formula Ic may then be obtained by treatment of XIII with a fluorinating agent such as SF₄/HF or F₃S—N(CH₂CH₂OMe)₂ followed by ester hydrolysis. 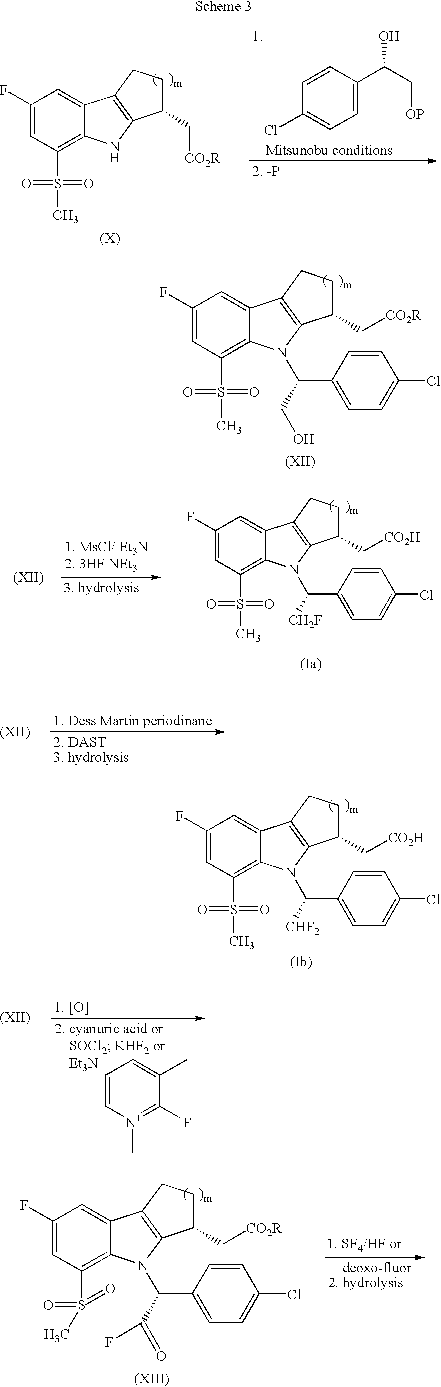 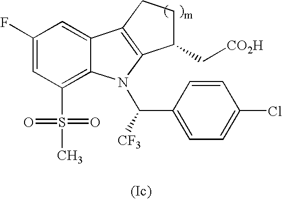
Assays for Determining Biological Activity

Compounds of formula I can be tested using the following assays to determine their prostanoid antagonist or agonist activity in vitro and in vivo and their selectivity. The prostaglandin receptor activities demonstrated are DP, EP₁, EP₂, EP₃, EP₄, FP, IP, TP and CRTH2.

Stable Expression of Prostanoid Receptors in the Human Embryonic Kidney (HEK) 293(ebna) Cell Line

Prostanoid receptor and CRTH2 cDNAs corresponding to full length coding sequences are subcloned into the appropriate sites of mammalian expression vectors and transfected into HEK 293(ebna) cells. HEK 293(ebna) cells expressing the individual cDNAs are grown under selection and individual colonies are isolated after 2-3 weeks of growth using the cloning ring method and subsequently expanded into clonal cell lines.

Prostanoid Receptor Binding Assays

HEK 293(ebna) cells are maintained in culture, harvested and membranes are prepared by differential centrifugation, following lysis of the cells in the presence of protease inhibitors, for use in receptor binding assays. Prostanoid receptor binding assays are performed in 10 mM MES/KOH (pH 6.0) (EPs, FP and TP) or 10 mM HEPES/KOH (pH 7.4) (DP, CRTH2 and IP), containing 1 mM EDTA, 10 mM divalent cation and the appropriate radioligand. The reaction is initiated by addition of membrane protein. Ligands are added in dimethylsulfoxide which is kept constant at 1% (v/v) in all incubations. Non-specific binding is determined in the presence of 1 μM of the corresponding non-radioactive prostanoid. Incubations are conducted for 60 min at room temperature or 30° C. and terminated by rapid filtration. Specific binding is calculated by subtracting non specific binding from total binding. The residual specific binding at each ligand concentration is calculated and expressed as a function of ligand concentration in order to construct sigmoidal concentration-response curves for determination of ligand affinity.

Prostanoid Receptor Agonist and Antagonist Assays

Whole cell second messenger assays measuring stimulation (EP₂, EP₄, DP and IP in HEK 293(ebna) cells) or inhibition (EP₃ in human erythroleukemia (HEL) cells) of intracellular cAMP accumulation or mobilization of intracellular calcium (EP₁, FP and TP in HEK 293(ebna) cells stably transfected with apo-aequorin) are performed to determine whether receptor ligands are agonists or antagonists. For cAMP assays, cells are harvested and resuspended in HBSS containing 25 mM HEPES, pH 7.4. Incubations contain 100 μM RO-20174 (phosphodiesterase type IV inhibitor, available from Biomol) and, in the case of the EP₃ inhibition assay only, 15 μM forskolin to stimulate cAMP production. Samples are incubated at 37° C. for 10 min, the reaction is terminated and cAMP levels are then measured. For calcium mobilization assays, cells are charged with the co-factors reduced glutathione and coelenterazine, harvested and resuspended in Ham's F12 medium. Calcium mobilization is measured by monitoring luminescence provoked by calcium binding to the intracellular photoprotein aequorin. Ligands are added in dimethylsulfoxide which is kept constant at 1% (v/v) in all incubations. For agonists, second messenger responses are expressed as a function of ligand concentration and both EC₅₀ values and the maximum response as compared to a prostanoid standard are calculated. For antagonists, the ability of a ligand to inhibit an agonist response is determined by Schild analysis and both K_{B} and slope values are calculated.

Prevention of PGD2 or Allergen Induced Nasal Congestion in Allergic Sheep

Animal preparation: Healthy adult sheeps (18-50 kg) are used. These animals are selected on the basis of a natural positive skin reaction to an intradermal injection of Ascaris suum extract.

Measurements of nasal congestion: The experiment is performed on conscious animals. They are restained in a cart in a prone position with their heads immobilized. Nasal airway resistance (NAR) is measured using a modified mask rhinometry technique. A topical anaesthesia (2% lidocaine) is applied to the nasal passage for the insertion of a nasotracheal tube. The maximal end of the tube is connected to a pneumotachograph and a flow and pressure signal is recorded on an oscilloscope linked to a computer for on-line calculation of NAR. Nasal provocation is performed by the administration of an aerosolized solution (10 puffs/nostril). Changes in the NAR congestion are recorded prior to and for 60-120 minutes post-challenge.

Prevention of PGD2 and Allergen Induced Nasal Obstruction in Cynomolgus Monkey

Animal preparation: Healthy adult male cynomologus monkeys (4-10 kg) are used. These animals are selected on the basis of a natural positive skin reaction to an intradermal injection of Ascaris suum extract. Before each experiment, the monkey selected for a study is fasted overnight with water provided at libitum. The next morning, the animal is sedated with ketamine (10-15 mg/kg i.m.) before being removed from its home cage. It is placed on a heated table (36° C.) and injected with a bolus dose (5-12 mg/kg i.v.) of propofol. The animal is intubated with a cuffed endotracheal tube (4-6 mm I.D.) and anaesthesia is maintained via a continuous intravenous infusion of propofol (25-30 mg/kg/h). Vital signs (heart rate, blood pressure, respiratory rate, body temperature) are monitored throughout the experiment.

Measurements of nasal congestion: A measurement of the animal respiratory resistance is taken via a pneumotachograph connected to the endotracheal tube to ensure that it is normal. An Ecovision accoustic rhinometer is used to evaluate nasal congestion. This technique gives a non-invasive 2D echogram of the inside of the nose. The nasal volume and the minimal cross-sectional area along the length of the nasal cavity are computed within 10 seconds by a laptop computer equipped with a custom software (Hood Laboratories, Mass, U.S.A.). Nasal challenge is delivered directly to the animal's nasal cavity (50 μL volume). The changes in nasal congestion are recorded prior to and for 60-120 minutes post-challenge. If nasal congestion occurs, it will translate into a reduction in the nasal volume.

Pulmonary Mechanics in Trained Conscious Squirrel Monkeys

The test procedure involves placing trained squirrel monkeys in chairs in aerosol exposure chambers. For control purposes, pulmonary mechanics measurements of respiratory parameters are recorded for a period of about 30 minutes to establish each monkey's normal control values for that day. For oral administration, compounds are dissolved or suspended in a 1% methocel solution (methylcellulose, 65HG, 400 cps) and given in a volume of 1 mL/kg body weight. For aerosol administration of compounds, a DeVilbiss ultrasonic nebulizer is utilized. Pretreatment periods vary from 5 minutes to 4 hours before the monkeys are challenged with aerosol doses of either PGD2 or Ascaris suum antigen; 1:25 dilution.

Following challenge, each minute of data is calculated by computer as a percent change from control values for each respiratory parameter including airway resistance (R_{L}) and dynamic compliance (C_{dyn}). The results for each test compound are subsequently obtained for a minimum period of 60 minutes post challenge which are then compared to previously obtained historical baseline control values for that monkey. In addition, the overall values for 60 minutes post-challenge for each monkey (historical baseline values and test values) are averaged separately and are used to calculate the overall percent inhibition of mediator or Ascaris antigen response by the test compound. For statistical analysis, paired t-test is used. (References: McFarlane, C. S., et al., Prostaglandins, 28, 173-182 (1984) and McFarlane, C. S. et al., Agents Actions, 22, 63-68 (1987).)

Prevention of Induced Bronchoconstriction in Allergic Sheep

Animal Preparation: Adult sheep with a mean weight of 35 kg (range, 18 to 50 kg) are used. All animals used meet two criteria: a) they have a natural cutaneous reaction to 1:1,000 or 1:10,000 dilutions of Ascaris suum extract (Greer Diagnostics, Lenois, N.C.); and b) they have previously responded to inhalation challenge with Ascaris suum with both an acute bronchoconstriction and a late bronchial obstruction (W. M. Abraham et al., Ann. Rev. Resp. Dis., 128, 839-44 (1983)).

Measurement of Airway Mechanics: The unsedated sheep are restrained in a cart in the prone position with their heads immobilized. After topical anesthesia of the nasal passages with 2% lidocaine solution, a balloon catheter is advanced through one nostril into the lower esophagus. The animals are then intubated with a cuffed endotracheal tube through the other nostril using a flexible fiberoptic bronchoscope as a guide. Pleural pressure is estimated with the esophageal balloon catheter (filled with one mL of air), which is positioned such that inspiration produces a negative pressure deflection with clearly discernible cardiogenic oscillations. Lateral pressure in the trachea is measured with a sidehole catheter (inner dimension, 2.5 mm) advanced through and positioned distal to the tip of the nasotracheal tube. Transpulmonary pressure, the difference between tracheal pressure and pleural pressure, is measured with a differential pressure transducer (DP45; Validyne Corp., Northridge, Calif.). For the measurement of pulmonary resistance (R_{L}), the maximal end of the nasotrachel tube is connected to a pneumotachograph (Fleisch, Dyna Sciences, Blue Bell, Pa.). The signals of flow and transpulmonary pressure are recorded on an oscilloscope (Model DR-12; Electronics for Medicine, White Plains, N.Y.) which is linked to a PDP-11 Digital computer (Digital Equipment Corp., Maynard, Mass.) for on-line calculation of R_{L} from transpulmonary pressure, respiratory volume obtained by integration and flow. Analysis of 10-15 breaths is used for the determination of R_{L}. Thoracic gas volume (V_{tg}) is measured in a body plethysmograph, to obtain specific pulmonary resistance (SR_{L}=R_{L}·V_{tg}).

The following examples are provided to illustrate the invention and are not to be construed as limiting the scope of the invention in any manner. In the examples, unless otherwise stated,
all the end products of the formula I were analyzed by NMR, TLC and elementary analysis or mass spectroscopy;
intermediates were analyzed by NMR and TLC;
most compounds were purified by flash chromatography on silica gel, recrystallization and/or swish (suspension in a solvent followed by filtration of the solid);
the course of reactions was followed by thin layer chromatography (TLC) and reaction times are given for illustration only;
the enantiomeric excess was measured on normal phase HPLC with a chiral column: ChiralPak AD; 250×4.6 mm.

### EXAMPLE 1

### [(3R)-4-[(1S)-1-(4-chlorophenyl)ethyl]-7-fluoro-5-(methylsulfonyl)-1,2,3,4-tetrahydro-cyclopenta[b]indol-3-yl]acetic acid and sodium salt

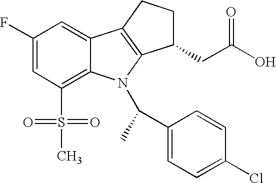 Step 1: To (S)-2-methyl-CBS-oxazaborolidine (Aldrich or Callery Chemical Co, 1M in toluene, 1 eq.) at −45° C. was added BH₃.Me₂S (1.06 eq.). To the previous solution was added at −30° C. a 1M dichloromethane solution of 4′-chloroacetophenone. After completion of the reaction, excess MeOH was added followed by 1N HCl. After warming up to room temperature, the resulting mixture was filtered through a pad of celite and silica gel using 30% EtOAc in hexane. The solvent was removed under reduced pressure and the resulting oil was purified by flash chromatography (20% EtOAc in hexane) to afford (1R)-1-(4-chlorophenyl)ethanol (ee ca. 98%).
Step 2: To a solution of [(3R)-5-bromo-7-fluoro-1,2,3,4-tetrahydrocyclopenta[b]indol-3-yl]acetic acid (see Reference Example 4) in DMF (250 mL) was added Cs₂CO₃ (1.3 eq.) followed by MeI (1.2 eq.) The reaction mixture was stirred for 6 h at room temperature and then diluted with 1:1 hexane/EtOAc. The insoluble material was removed by filtration through a pad of silica gel and the filtrate was concentrated under reduced pressure to yield methyl [(3R)-5-bromo-7-fluoro-1,2,3,4-tetrahydrocyclopenta[b]indol-3-yl]acetate as a brown solid. ¹HNMR (500 MHz, acetone-d6) δ 9.90 (1H, bs), 7.16 (1H, d, J=9.5 Hz), 7.12 (1H, d, J=9.0 Hz), 3.71 (3H, s), 3.64 (1H, m), 2.70-2.95 (5H, m), 2.60 (1H, dd, J=16, 8 Hz), 2.22 (1H, m).
Step 3: To a solution of compound of Step 2 in anhydrous DMSO were added sodium methanesulphinate (2.0 eq.) and copper iodide (2.0 eq.). The resulting mixture was heated at 110° C. under nitrogen using mechanical stirring. After a period of 18 h, additional sodium methanesulphinate (2.0 eq.) and copper iodide (2.0 eq.) were added. After a period of 5 h, the reaction mixture was cooled to room temperature, poured into EtOAc and stirred for 18 h and then filtered over celite. The celite was washed with EtOAc and the filtrate was washed 3 times with water. The solvent was removed under reduced pressure and the crude mixture was purified by flash chromatography using 10% CH₂Cl₂-20% EtOAc in hexane to provide methyl [(3R)-7-fluoro-5-(methylsulfonyl)-1,2,3,4-tetrahydrocyclopenta[b]indol-3-yl]acetate.
Step 4: To a solution of compound of Step 3, triphenylphosphine (1.5 eq.), and (1R)-1-(4-chlorophenyl)ethanol (1.5 eq.) in THF (0.075M) was added a THF solution (4.7 M) of di-tert-butylazodicarboxylate (1.5 eq.) over 10 min. The reaction mixture was stirred at room temperature for 30 min. and concentrated. The crude product was purified by flash chromatography using 25% EtOAc in hexane to provide methyl [(3R)-4[(1S)-1-(4-chlorophenyl)ethyl]-7-fluoro-5-(methylsulfonyl)-1,2,3,4-tetrahydrocyclopenta[b]indol-3-yl]acetate, which contains some by products and was used as such for the next step.
Step 5: To a solution of compound of Step 4 in a 1:1 THF-MeOH mixture (0.1M) was added NaOH 1M (4.4 eq.). After a period of 2 h, the reaction mixture was acidified with 1N HCl and extracted with EtOAc. The crude product was purified by flash chromatography with 30% EtOAc in hexane followed by 1% AcOH in 30% EtOAc-hexane. After evaporation of the solvents, the compound was stirred in 2% EtOAc-hexane and filtered. The compound was purified again by flash chromatography was using 2% AcOH in 2% EtOAc-toluene to provide a solid which was stirred for 18 h in hexane and filtered. ¹HNMR(500 MHz, acetone-d₆) δ 10.80 (1H, bs), 7.70 (1H, m), 7.60 (1H, m), 7.30 (2H, d), 6.95 (2H, d), 6.90 (1H, m), 3.40 (3H, s), 3.05 to 2.15 (7H, m), 2.10 (3H, d).

To a suspension of the acid in water was added 1N aqueous NaOH (1 eq.). The resulting solution was lyophilized to provide the sodium salt as a white solid. ¹HNMR (500 MHz, DMSO-d₆) δ 7.55 (2H, m), 7.30 (2H, d), 6.85 (2H, d), 6.65 (1H, m), 3.40 (3H, s), 3.00 to 1.95 (7H, m), 2.05 (3H, d).

### EXAMPLE 2

### [(1R)-9-[(1S)-1-(4-chlorophenyl)ethyl]-6-fluoro-8-(methylsulfonyl)-2,3,4,9-tetrahydro-1H-carbazol-1-yl]acetic acid and sodium salt

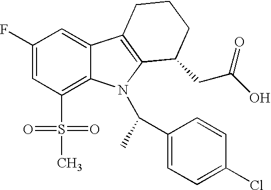 Step 1: To a suspension of 2-bromo-4-fluoroaniline in concentrated HCl (1.5M) at −10° C. was slowly added a 10.0M aqueous solution of NaNO₂ (1.1 eq). The mixture was stirred at 0° C. for 2.5 hrs. A cold (−30° C.) solution of SnCl₂ (3.8M) in concentrated HCl was then slowly added while maintaining the internal temperature below 10° C. The resulting mixture was stirred mechanically for 20 min at 0, then at room temperature for 1 hr. The thick slurry was filtered and the solid was air dried overnight. The solid was resuspended in cold HCl and filtered again. The dried material was suspended in Et₂O, stirred for 10 min, filtered and air dried overnight to give 2-(2-bromo-4-fluorophenyl)hydrazinium chloride as a beige solid.
Step 2: To a suspension of 2-(2-bromo-4-fluorophenyl)hydrazinium chloride (1 eq) in AcOH (0.5M) was added ethyl (2-oxocyclohexyl)acetate (1 eq). The mixture was stirred at reflux for 16 hrs, cooled and AcOH was removed by evaporation under reduced pressure. The residue was diluted with EtOAc and washed with water and saturated aqueous NaHCO₃. The organic layer was dried over Na₂SO₄ and concentrated. The residue was then purified on a pad of silica gel, eluting with toluene. The filtrate was concentrated and stirred in hexanes to give, after filtration, (+/−)-ethyl (8-bromo-6-fluoro-2,3,4,9-tetrahydro-1H-carbazol-1-yl)acetate as a white solid. MS (+APCI) m/z 354.2 (M+H)⁺
Step 3: To a solution of compound of Step 2 (1 eq) in anhydrous DMSO (0.28M) were added sodium methanesulphinate (3 eq) and copper iodide (3 eq). N₂ was bubbled into the mixture for 5 min and the reaction was then stirred at 100° C. under N₂ atmosphere. After 12 hrs, more sodium methanesulphinate (2 eq) and copper iodide (2 eq) were added. The mixture was stirred for a further 12 hrs at 100° C., cooled, diluted with EtOAc and 1N HCl was added to acidify the mixture. The suspension was stirred for 30 min and filtered through celite. The filtrate was washed with water, dried over Na₂SO₄ and concentrated. The residue was filtered through a pad of silica gel, eluting first with toluene to remove the non-polar impurities and then with a 2:1 mixture of hexanes/EtOAc to elute the desired product. The filtrate from the elution with the mixture of hexanes/EtOAc was concentrated to give (+/−)-ethyl [6-fluoro-8-(methylsulfonyl)-2,3,4,9-tetrahydro-1H-carbazol-1-yl]acetate as a pale yellow solid. MS (−APCI) m/z 352.1 (M−H)⁻
Step 4: The racemic mixture from step 3 was resolved by preparative HPLC on a chiralpak AD preparative column eluted with a mixture of 15% iPrOH in hexane. The more polar enantiomer (longer retention time) was identified as ethyl [(1R)-6-fluoro-8-(methylsulfonyl)-2,3,4,9-tetrahydro-1H-carbazol-1-yl]acetate based on the activity of the final product.
Step 5: To a solution of compound of Step 4 (1 eq), triphenylphosphine (1.5 eq) and (1R)-1-(4-chlorophenyl)ethanol from step 1 example 1 (1.5 eq) in THF (0.175M) was added a solution of di-tert-butyl azodicarboxylate (2.1 M in THF, 1.5 eq) over a 10 min period. The mixture was stirred at room temperature for 2 hr and concentrated. The residue was purified by silica gel flash chromatography, eluting with 7% EtOAc in toluene to give ethyl [(1R)-9-[(1S)-1-(4-chlorophenyl)ethyl]-6-fluoro-8(methylsulfonyl)-2,3,4,9-tetrahydro-1H-carbazol-1-yl]acetate (˜90% pure) which was used as such for the next reaction.
Step 6: To a solution of compound of Step 5 in a 2:1 mixture of THF and methanol (0:1M) was added 1N aqueous LiOH (3 eq). The mixture was stirred at room temperature for 2 hr, AcOH was added and the solvent was removed by evaporation. The residue was taken up in EtOAc/H₂O and the organic layer was washed with brine, dried over Na₂SO₄, filtered and concentrated. The residue was swished in 30% EtOAc in hexane, and the product was suspended in diethyl ether and sonicated for 45 min, filtered, and dried under high vacuum at 50° C. for 24 hr to give the title acid as a white solid. MS (−APCI) m/z 462.1 (M−H)⁻

To a suspension of the acid in methanol was added 1N aqueous NaOH (1 eq.). The methanol was evaporated and water was added. The resulting solution was lyophilized to provide the sodium salt as a white solid. ¹HNMR(500 MHz, acetone-d6) δ 7.60 (1H, m), 7.40 (1H, m), 7.15 (2H, d), 6.70 (1H, m), 6.55 (2H, d), 3.30 (3H, s), 3.05 (1H, m), 2.65 (1H, m), 2.55 (1H, m), 2.45 (1H, m), 2.35 (1H, m), 2.20 (3H, d), 1.85 (1H, m), 1.75 (1H, m), 1.60 (1H, m), 1.10 (1H, m).

Alternatively (+/−) ethyl [6-fluoro-8-(methylsulfonyl)-2,3,4,9-tetrahydro-1H-carbazol-1-yl]acetate was used for the alkylation reaction in step 5 to give a mixture of 2 diastereomers: ethyl [(1R)-9-[(1S)-1-4-chlorophenyl)ethyl]-6-fluoro-8-(methylsulfonyl)-2,3,4,9-tetrahydro-1H-carbazol-1-yl]acetate and ethyl [(1S)-9-[(1S)-1-(4-chlorophenyl)ethyl]-6-fluoro-8-methylsulfonyl)-2,3,4,9-tetrahydro-1H-carbazol-1-yl]acetate.

The above diastereomeric mixture (1 eq) was dissolved in a 3.5/1 mixture of THF/MeOH (0.25M) and cooled at 0° C. Aqueous LiOH 1N (1 eq) was slowly added and the mixture was stirred at 0° C. for 12 h or until almost complete hydrolysis of ethyl [(1R)-9-[(1S)-1-(4-chlorophenyl)ethyl]-6-fluoro-8-(methylsulfonyl)-2,3,4,9-tetrahydro-1H-carbazol-1-yl]acetate, the other diastereomer was only slightly hydrolyzed under these conditions. AcOH was added and the solvent was removed by evaporation. The residue was taken up in EtOAc/H₂O and the organic layer was washed with brine, dried over Na₂SO₄, filtered and concentrated. Ethyl [(1S)-9-[(1S)-1-(4-chlorophenyl)ethyl]-6-fluoro-8-(methylsulfonyl)-2,3,4,9-tetrahydro-1H-carbazol-1-yl]acetate and [(1R)-9-[(1S)-1-(4-chlorophenyl)ethyl]-6-fluoro-8-(methylsulfonyl)-2,3,4,9-tetrahydro-1H-carbazol-1-yl]acetic acid were separated by flash chromatography eluting with 40% EtOAc in hexanes containing 1% AcOH to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid with de>90% which was swished in 30% EtOAc in hexane to give the desired compound as a white solid with de>95%.

### EXAMPLE 3

### [(1R)-9-[(1R)-1-(4-chlorophenyl)-2-fluoroethyl]-6-fluoro-8-(methylsulfonyl)-2,3,4,9-tetrahydro-1H-carbazol-1-yl]acetic acid

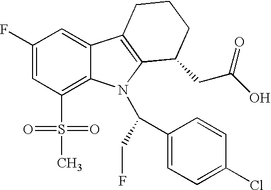 Step 1: To a 1:1 mixture of H₂O:tBuOH (0.1M) was added AD mix-α (Aldrich-Sigma, 1.4 g/mmol of olefin) and the mixture was stirred at rt until the reagent turned in solution, and then cooled to 0° C. 1-Chloro-4-vinylbenzene (1 eq) was added in one portion and the reaction mixture was stirred at 0° C. for 16 hrs. Solid sodium sulfite (1.6 g/mmol of olefin) was added. The mixture was stirred at rt for 30 minutes and then extracted with EtOAc, the combined organic layers were dried over Na₂SO₄ and concentrated to give (1S)-1-(4-chlorophenyl)ethane-1,2-diol which was used as such for the next step.
Step 2: To a solution of (1S)-1-(4-chlorophenyl)ethane-1,2-diol (1 eq) in CH₂Cl₂ (0.2M) was added imidazole (1.5 eq) and then tert-butyldimethylsilyl chloride (1 eq) portion wise. The reaction mixture was stirred at rt for 1 hr, brine was added and the reaction mixture was extracted with CH₂Cl₂, the combined organic layers were dried over Na₂SO₄ and concentrated. The residue was purified by flash chromatography on silica gel eluted with gradient from 10% EtOAc/hexane to 30% EtOAc/hexane to give (1S)-2-{[tert-butyl(dimethyl)silyl]oxy}-1-(4-chlorophenyl)ethanol.
Step 3: To a solution of the acid of Example 2 ([α]_{D}=−226° in MeOH) in MeOH (0.1M) was added 10% palladium on carbon (10% wt/wt). A stream of N₂ was bubbled through the mixture for 5 min. The reaction mixture was stirred at rt under H₂ atmosphere (balloon) for 24 hrs and filtered through a celite pad eluted with CH₂Cl₂. The solvents were removed by evaporation under reduced pressure and the residue was swished in MeOH to give methyl [(1R)-6-fluoro-8-(methylsulfonyl)-2,3,4,9-tetrahydro-1H-carbazol-1-yl]acetate. To a solution of this methyl ester (1 eq), triphenylphosphine (1.5 eq) and compound of Step 2 (1.5 eq) in THF (0.2M) was added a solution of di-tert-butyl azodicarboxylate (1M in THF, 1.5 eq) over a 20 min period. The mixture was stirred at room temperature for 2 hr and concentrated. The residue was purified by silica gel flash chromatography eluted with 10% EtOAc in toluene to give methyl [(1R)-9-[(1R)-2-{[tert-butyl(dimethyl)silyl]oxy}-1-(4-chlorophenyl)ethyl]-6-fluoro-8-methylsulfonyl)-2,3,4,9-tetrahydro-1H-carbazol-1-yl]acetate (˜90% pure) which was used as such for the next reaction.
Step 4: To a solution of compound of Step 3 (1 eq) in THF (0.1 M) was added 1M/THF tetrabutylammonium fluoride (1.5 eq). The reaction mixture was stirred at rt for 1 h and saturated aqueous NH₄Cl was added. The reaction mixture was extracted with EtOAc, and the combined organic layers were dried over Na₂SO₄ and concentrated. The residue was purified by flash chromatography on silica gel eluted with gradient from 30% EtOAc/hexane to 50% EtOAc/hexane to give methyl [(1R)-9-[(1R)-1-(4-chlorophenyl)-2-hydroxyethyl]-6-fluoro-8-methylsulfonyl)-2,3,4,9-tetrahydro-1H-carbazol-1-yl]acetate.
Step 5: To a solution of compound of Step 4 (1 eq) in CH₂Cl₂ (0.06M) at 0° C. was added triethylamine (2 eq) followed by methanesulfonyl chloride (1.5 eq). The reaction mixture was stirred at 0° C. for 30 minutes and quenched with aqueous saturated NaHCO₃. The reaction mixture was extracted with CH₂Cl₂, and the combined organic layers were dried over Na₂SO₄ and concentrated. The resulting mesylate (1 eq) was dissolved in triethylamine trihydrofluoride (23 eq) and stirred at 200° C. for 5 min at high power in microwave. The reaction mixture was poured into aqueous saturated NaHCO₃ and extracted with EtOAc. The combined organic layers were washed with brine, dried over Na₂SO₄ and concentrated. The residue was purified by flash chromatography on silica gel eluted with gradient from 10% EtOAc/hexane to 30% EtOAc/hexane to give methyl [(1R)-9-[(1R)-1-(4-chlorophenyl)-2-fluoroethyl]-6-fluoro-8-(methylsulfonyl)-2,3,4,9-tetrahydro-1H-carbazol-1-yl]acetate.
Step 6: To a solution of compound of Step 5 (1 eq) in a 3.5/1 mixture of THF/MeOH (0.25M) at 0° C. was slowly added aqueous LiOH 1N (1 eq) and the mixture was stirred at 0° C. for 16 h or until almost complete hydrolysis of the ester; under these conditions, the minor diastereomer has a much slower rate of hydrolysis. AcOH was added and the solvents were removed in vacuo. The residue was taken up in EtOAc/H₂O and the organic layer was washed with brine, dried over Na₂SO₄, filtered and concentrated. To remove the unreacted methyl ester and other impurities, the residue was filtered through a pad of silica gel eluting first with 10% EtOAc/toluene and then with 60% EtOAc/toluene containing 1% of AcOH to elute the desired acid. The residue was swished in 30% EtOAc/hexane and dried under high vacuum at 50° C. for 16 hr to give the desired compound as a white solid with de and ee>95% (checked by chiral HPLC) MS (+APCI) m/z 482.1 (M+H)⁺. [α]_{D}=−217° in MeOH

### EXAMPLE 4

### [(1R)-9-[(1R)-1-(4-chlorophenyl)-2,2-difluoroethyl]-6-fluoro-8-(methylsulfonyl)-2,3,4,9-tetrahydro-1H-carbazol-1-yl]acetic acid

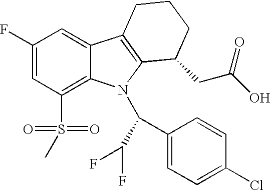 Step 1: To a solution of compound of Example 3, Step 4 (1 eq) in CH₂Cl₂ (0.1 M) was added Dess-Martin Periodinane (1.5 eq). The reaction mixture was stirred at rt for 1 h, H₂O (10 eq) was added and the reaction mixture was stirred for 30 minutes and filtered through a silica gel pad eluted with 50% EtOAc/hexane and concentrated. The residue was purified by flash chromatography on silica gel eluted with gradient from 10% EtOAc/hexane to 50% EtOAc/hexane to give methyl [(1R)-9-[(1R)-1-(4-chlorophenyl)-2-oxoethyl]-6-fluoro-8-(methylsulfonyl)-2,3,4,9-tetrahydro-1H-carbazol-1-yl]acetate.
Step 2: To a solution of compound of Step 1 (1 eq) in CH₂Cl₂ (0.08 M) at −78° C. was added (N,N-diethylamino)sulphur trifluoride (1.5 eq). The reaction mixture was slowly warmed to 0° C. and stirred over week-end at 5° C. The mixture was poured into aqueous saturated NaHCO₃ and extracted with EtOAc. The combined organic layers were dried over Na₂SO₄ and concentrated. The residue was purified by flash chromatography on silica gel eluted with gradient from 10% EtOAc/hexane to 30% EtOAc/hexane to give methyl [(1R)-9-[(1R)-1-(4-chlorophenyl)-2,2-difluoroethyl]-6-fluoro-8-(methylsulfonyl)-2,3,4,9-tetrahydro-1H-carbazol-1-yl]acetate.
Step 3: Starting from compound of Step 2, the title compound was synthesized following the procedure described in Step 6 of Example 3. MS (-APCI) m/z 480.0 (M−F)⁻. [α]_{D}=−237° in MeOH

### REFERENCE EXAMPLE 1

### Preparation of (+/−)-(7-fluoro-1,2,3,4-tetrahydrocyclopenta[b]indol-3-yl)acetic acid ethyl ester

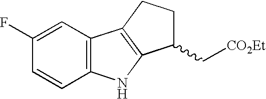

A mixture of ethyl (2-oxocyclopentyl)acetate (1.0 eq.), 2-bromo-4-fluoroaniline (1.05 eq.), and triethylphosphite (1.20 eq.) is treated with 85% phosphoric acid (4 mol %, 0.04 eq.) and the reaction mixture is then warmed to 60° C. under nitrogen. After 7 h the reaction mixture is allowed to cool to room temperature (25-20° C.) and is stirred into a 10/90 volume ratio of triethylamine/cyclohexane (10 L/Kg of the cyclopentylacetate). Water (5 L/Kg of the cyclopentylacetate) is added to the mixture, and the mixture is stirred for 15 minutes. The layers are separated and the organic phase is washed twice with water (2×5 L/kg of cyclopentylacetate), then distilled at constant volume under house vacuum at room temp with a half volume (5 L/kg of cyclopentylacetate) of cyclohexane to remove residual water. Finally, the solvent is switched to dimethylacetamide (DMAC, 1 L/mole cyclopentylacetate) for the cyclization step.

To the above reaction mixture is added triethylamine (2 eq.). Tri-o-tolylphosphine (12 mol %, 0.12 eq.) and palladium acetate (3 mol %, 0.03 eq.) are charged and the solution is degassed with three nitrogen/vacuum purges. The solution is heated at 90° C. for 6 h, then cooled to 20° C. and reverse quenched into a stirred biphasic solution made of a 10 wt % KH₂PO₄ aqueous solution (10 L/kg of cyclopentylacetate) and MTBE (10 L/kg of cyclopentylacetate). The mixture is stirred for 15 minutes and layers are separated. The organic phase is washed twice with water (2×5 L/kg of cyclopentylacetate). The organic layer is then filtered through a pad of solka-floc and concentrated under house vacuum at room temp. The solution is then switched to DMF (2.5 L/kg of cyclopentylacetate) and is used as is for the next step (enzymatic resolution).

### REFERENCE EXAMPLE 2

### Preparation of ethyl (3R)-(7-fluoro-1,2,3,4-tetrahydrocyclopenta[b]indol-3-yl)acetate

Enzyme Resolution

To a reaction vessel equipped with a stirrer, temperature control, and pH control (with NaOH (3.8N) addition via a peristaltic pump), add a volume of the mixture from Reference Example 1 to provide 100 g of the racemate, and Pseudomonas fluorescens Lipase AK-AF (Amano 20, Lot # LAKAF1152102, 840 kU of enzyme/100 g racemate) in buffer (pH 8.0, 0.2M dibasic potassium phosphate in deionized water, sufficient volume to make up 1 liter reaction mixture). The reaction is carried out using the following reactor settings: pH=8.0, temperature=28° C., stir rate=400 RPM. A typical optical purity of 95% ee of the desired ester is obtained at 49% conversion at approximately 24 hours into the reaction. Greater than 99% ee of the desired ester is obtained after 38 hours reaction time.

### REFERENCE EXAMPLE 3

### Preparation of (3R)-(7-fluoro-1,2,3,4-tetrahydrocyclopenta[b]indol-3-yl)acetic acid, dicyclohexylamine salt

Once the resolution described in Reference Example 2 is complete (e.e≧98%), ½ volume of acetonitrile is added to the mixture followed by the addition of ½ volume of methyl t-butyl ether (MTBE), and solka-floc (15 wt %). The reaction mixture is stirred at room temperature for ca. 1 hour and filtered. The pad of solka-floc is rinsed with ½ volume of MTBE. The solution is pumped back into the vessel and is further diluted with ½ volume of MTBE. A ½ volume of 4% aq. sodium hydroxide (4 g/L; 0.1 N) is added, and the biphasic mixture is stirred for ca. 15 min, allowed to settle and the layers are separated. The organic layer is then washed twice with ½ volume of a 5 wt % aqueous sodium bicarbonate solution (50 g/L, 2×½ volume). DMAc (2.5 L/kg indole ester) is added to the organic layer along with n-heptane (2.5 L/kg of indole ester) and 5N aq. NaOH (0.76 L/kg indole ester, 1 equivalent) is added over 5 min at r.t. The biphasic mixture is stirred for 2 hours and allowed to settle. Layers are separated and the organic is washed with water (1.5 L/kg indole ester). Combined basic aqueous DMAc solution is pumped back into the vessel. MTBE (7.5 L/kg of indole ester) is added and the aqueous is neutralized at r.t. to pH˜1-2 with 5% aqueous HCl (ca. 0.6 N, 8.5 L/kg of indole ester) over stirring and cooling. Layers are separated and the organic is washed twice with water (2×3.5 L/kg of indole ester). The MTBE solution is filtered (10 μm), concentrated and switched to acetonitrile until KF≦500. The final total volume is adjusted to ca. 6.5 L/kg of indole ester. The solution is heated to +50° C. and dicyclohexylamine (DCHA, 0.16 equivalents) is added in one portion and the batch is aged for 1 hour at +50° C. Remaining DCHA (0.39 equivalents) is added over 1 hour. The mixture is aged at +50° C. for ca. 1 h, allowed to cool to r.t, and further aged for ca. 10 h. The batch is filtered, rinsed with acetonitrile (1 L/kg of indole ester) and dried in the oven at +40° C. for 24 h.

### REFERENCE EXAMPLE 4

### Preparation of (3R)-(5-bromo-7-fluoro-1,2,3,4-tetrahydrocyclopenta[b]indol-3-yl)acetic acid

A solution of the DCHA salt (Reference Example 3, 1 equivalent) in dichloromethane (10 L/Kg DCHA salt) containing pyridine (2 eq.) is added to a solution of bromine (2.5 eq.) in dichloromethane (3 L/Kg DCHA salt) that is cooled to −15 to −10° C. The rate of addition is adjusted such that the temperature is kept between −15 and −10° C. After completion of the addition, the reaction mixture is aged for one hour at −15° C. The reaction mixture (loose slurry, kept at −15° C.) is added to a slurry of zinc dust (2.5 eq.) in dichloromethane (3 L/Kg DCHA salt) containing acetic acid (3 eq.) that is cooled to ca. −10° C. The rate of addition is adjusted such that the temperature is kept between −10° C. and −5° C. After completion of the addition, the batch is warmed to r.t., aged for 1 hour, and concentrated down at atmospheric pressure to about ⅓ of its initial volume. Water (8 L/Kg DCHA salt) is added followed by the addition of MTBE (8 L/Kg DCHA salt) to precipitate the salts byproducts. Distillation is resumed and run at constant volume by adding 1 volume of MTBE (8 L/Kg DCHA salt). The distillation is stopped when the final volume of the solution is ca. 21.6 L/Kg DCHA salt. The reaction mixture is then filtered. The cake is rinsed with MTBE (ca. 8 L/Kg DCHA salt) and the filtrate (MTBE/aqueous) is pumped back into the vessel. Layers are separated and the organic phase is washed with water (8 L/Kg DCHA salt). The MTBE solution is concentrated (in-line filtered) and switched to 2-propanol (2.2 L/Kg DCHA salt) to crystallize the product. Water (5.2 L/Kg DCHA salt) is added over 2 hours. The batch is aged for a couple hours, filtered and rinsed with 30/70 2-propanol/Water (1.7 L/Kg DCHA salt). Crystallized bromoacid is dried at +40° C.

What is claimed is:

1. A compound of formula I: 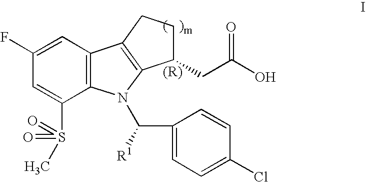
and pharmaceutically acceptable salts thereof, wherein m is 1 or 2, and R¹ is C₁₋₃alkyl optionally substituted with 1 to 5 halogen atoms.

2. A compound of claim 1 selected from [(3R)-4-[(1S)-1-(4-chlorophenyl)ethyl]-7-fluoro-5-(methylsulfonyl)-1,2,3,4-tetrahydrocyclopenta[b]indol-3-yl]acetic acid and pharmaceutically acceptable salts thereof, [(1R)-9-[(1S)-1-(4-chlorophenyl)ethyl]-6-fluoro-8-(methylsulfonyl)-2,3,4,9-tetrahydro-1H-carbazol-1-yl]acetic acid and pharmaceutically acceptable salts thereof, [(1R)-9-[(1R)-1-(4-chlorophenyl)-2-fluoroethyl]-6-fluoro-8-methylsulfonyl)-2,3,4,9-tetrahydro-1H-carbazol-1-yl]acetic acid and pharmaceutically acceptable salts thereof, and [(1R)-9-[(1R)-1-(4-chlorophenyl)-2,2-difluoroethyl]-6-fluoro-8-(methylsulfonyl)-2,3,4,9-tetrahydro-1H-carbazol-1-yl]acetic acid and pharmaceutically acceptable salts thereof.

3. A pharmaceutical composition comprising a compound of claim 1 and a pharmaceutically acceptable carrier.

4. The composition of claim 3 further comprising a second active ingredient selected from an antihistamine, a leukotriene antagonist and a leukotriene biosynthesis inhibitor.

5. A method for the treatment of prostaglandin D2 mediated diseases which comprises administering to a patient in need of such treatment a therapeutically effective amount of a compound of claim 1.

6. A method for the treatment of nasal congestion which comprises administering to a patient in need of such treatment a therapeutically effective amount of a compound of claim 1.

7. A method for the treatment of allergic asthma which comprises administering to a patient in need of such treatment a therapeutically effective amount of a compound of claim 1.

8. A method for the treatment of allergic rhinitis which comprises administering to a patient in need of such treatment a therapeutically effective amount of a compound of claim 1.

9. A prostaglandin D2 receptor (DP receptor) antagonist pharmaceutical composition comprising an acceptable antagonistic amount of a compound of formula I, as defined in claim 1 or 2, or a pharmaceutically acceptable salt thereof, in association with a pharmaceutically acceptable carrier.

10. Use of a compound of formula I, as defined in claim 1 or 2, or a pharmaceutically acceptable salt thereof, in the manufacture of a medicament for treatment of prostaglandin D2 mediated diseases.

11. Use of a compound of formula I, as defined in claim 1 or 2, or a pharmaceutically acceptable salt thereof, in the manufacture of a medicament for treatment of nasal congestion, allergic asthma or allergic rhinitis.

12. A compound of formula I, as defined in claim 1 or 2, or a pharmaceutically acceptable salt thereof, for use in medical therapy.

13. A compound of formula I, as defined in claim 1 or 2, or a pharmaceutically acceptable salt thereof, for use in the treatment of prostaglandin D2 mediated diseases.

14. A compound of formula I, as defined in claim 1 or 2, or a pharmaceutically acceptable salt thereof, for use in the treatment of nasal congestion, allergic asthma or allergic rhinitis.
